SEQUENCE LISTING (43 sequences)

SEQ ID NO: 1 - protein, 7 aa, unidentified
ENLYFQG

SEQ ID NO: 2 - protein, 19 aa, synthetic construct
KRDHMVLLEFVTAAGITGT

SEQ ID NO: 3 - protein, 19 aa, synthetic construct
KRDHMVLHEFVTAAGITGT

SEQ ID NO: 4 - protein, 17 aa, synthetic construct
KRDHMVLHESVNAAGIT

SEQ ID NO: 5 - protein, 16 aa, synthetic construct
RDHMVLHEYVNAAGIT

SEQ ID NO: 6 - protein, 17 aa, synthetic construct
GDAVQIQEHAVAKYFTV

SEQ ID NO: 7 - protein, 17 aa, synthetic construct
GDTVQLQEHAVAKYFTV

SEQ ID NO: 8 - protein, 17 aa, synthetic construct
GETIQLQEHAVAKYFTE

SEQ ID NO: 9 - protein, 13 aa, synthetic construct
SRLEEELRRRLTE

SEQ ID NO: 10 - protein, 15 aa, synthetic construct
GLNDIFEAQKIEWHE

SEQ ID NO: 11 - protein, 4 aa, synthetic construct
EPEA

SEQ ID NO: 12 - protein, 26 aa, Unidentified
KRRWKKNFIAVSAANRFKKISSSGAL

SEQ ID NO: 13 - protein, 23 aa, synthetic construct
DIPATYEFTDGKHYITNEPIPPK

SEQ ID NO: 14 - protein, 13 aa, synthetic construct
GAPVPYPDPLEPR

SEQ ID NO: 15 - protein, 8 aa, synthetic construct
DYKDDDDK

SEQ ID NO: 16 - protein, 19 aa, synthetic construct
EELLSKNYHLENEVARLKK

SEQ ID NO: 17 - protein, 9 aa, Human Influenza Virus
YPYDVPDYA

SEQ ID NO: 18 - protein, 6 aa, synthetic construct
HHHHHH

SEQ ID NO: 19 - protein, 16 aa, Streptococcus pyogenes
TDKDMTITFTNKKDAE

SEQ ID NO: 20 - protein, 20 aa, synthetic construct
FIDTNNDGWIEGDELLLEEG

SEQ ID NO: 21 - protein, 10 aa, Homo sapiens
EQKLISEEDL

SEQ ID NO: 22 - protein, 18 aa, synthetic construct
TKENPRSNQEESYDDNES

SEQ ID NO: 23 - protein, 7 aa, synthetic construct
EEEEEEE

SEQ ID NO: 24 - protein, 7 aa, synthetic construct
RRRRRRR

SEQ ID NO: 25 - protein, 9 aa, Bos taurus
TETSQVAPA

SEQ ID NO: 26 - protein, 38 aa, synthetic construct
MDEKTTGWRGGHVVEGLAGELEQLRARLEHHPQGQREP

SEQ ID NO: 27 - protein, 13 aa, synthetic construct
DPIVMIDNDKPIT

SEQ ID NO: 28 - protein, 13 aa, Unidentified
STVPVAPPRRRRG

SEQ ID NO: 29 - protein, 5 aa, synthetic construct
GSHHW

SEQ ID NO: 30 - protein, 12 aa, synthetic construct
KLGDIEFIKVNK

SEQ ID NO: 31 - protein, 13 aa, Escherichia coli
SLAELLNAGLGGS

SEQ ID NO: 32 - protein, 8 aa, Homo sapiens
TQDPSRVG

SEQ ID NO: 33 - protein, 12 aa, synthetic construct
PDRVRAVSHWSS

SEQ ID NO: 34 - protein, 13 aa, Streptococcus pyogenes
AHIVMVDAYKPTK

SEQ ID NO: 35 - protein, 15 aa, Unidentified
KETAAAKFERQHMDS

SEQ ID NO: 36 - protein, 9 aa, synthetic construct
AWAHPQPGG

SEQ ID NO: 37 - protein, 9 aa, synthetic construct
AWRHPQFGG

SEQ ID NO: 38 - protein, 8 aa, synthetic construct
WSHPQFEK

SEQ ID NO: 39 - protein, 11 aa, synthetic construct
MASMTGGQQMG

SEQ ID NO: 40 - protein, 10 aa, synthetic construct
EVHTNQDPLD

SEQ ID NO: 41 - protein, 6 aa, synthetic construct
CCPGCC

SEQ ID NO: 42 - protein, 11 aa, vesicular stomatitis virus
YTDIEMNRLGK

SEQ ID NO: 43 - protein, 8 aa, synthetic construct
DLYDDDDK

(19) United States
(12) Patent Application Publication
(10) Pub. No.: US 2024/0280580 A1
(43) Pub. Date: Aug. 22, 2024

(54) PROTEIN AGGREGATION ASSAYS

(71) Applicant: Nuclera Ltd., Impington Cambridge (GB)

(72) Inventors: Michael Chun Hao CHEN, Impington Cambridge (GB); Marco MANNI, Impington Cambridge (GB); Gordon Ross MCINROY, Impington Cambridge (GB)

(21) Appl. No.: 18/588,757

(22) Filed: Feb. 27, 2024

Related U.S. Application Data
(63) Continuation-in-part of application No. PCT/GB2024/050050, filed on Jan. 10, 2024.

(30) Foreign Application Priority Data
Jan. 10, 2023 (GB) 2300350.2
Jan. 10, 2023 (GB) 2300351.0

(51) Int. Cl. G01N 33/68; G01N 21/77; G01N 33/58

(52) U.S. Cl. CPC G01N 33/6803; G01N 21/77; G01N 33/582; G01N 2021/7786

(57) ABSTRACT

The invention provided herein relates to methods for measuring protein aggregation and stability. The methods may be applied to characterising cell-free protein synthesis reactions.

### REFERENCE TO RELATED APPLICATIONS

This application is a continuation-in-part (CIP) application of International Patent Application No. PCT/GB2024/050050, filed on Jan. 10, 2024, which claims priority to UK Patent Application Nos. GB 2300350.2 and GB 2300351.0, both filed on Jan. 10, 2023. The entire contents of each of the above-referenced applications are hereby incorporated herein by reference.

### SEQUENCE LISTING

The instant application contains a Sequence Listing XML file which has been filed electronically in XML format, and is hereby incorporated by reference in its entirety. Said XML file, created on Feb. 15, 2024, is named “Nuclera P093-B37706.xml” and is 38,305 bytes in size.

### FIELD OF THE INVENTION

Provided herein are methods for improved expression and characterisation of proteins in cell-free systems.

### BACKGROUND

Proteins are biological macromolecules that maintain the structural and functional integrity of the cell, and many diseases are associated with protein malfunction. Protein purification is a fundamental step for analysing individual proteins and protein complexes and identifying interactions with other proteins, DNA or RNA. A variety of protein purification strategies exist to address desired scale, throughput and downstream applications. However, protein production can be challenging for many reasons. One major challenge is finding a suitable expression system, for example sourced from mammalian, bacterial, fungal, or plant cells. This can take months of work.

Cell-free protein synthesis (CFPS), also known as coupled or uncoupled in-vitro transcription and translation, is the production of peptides or proteins using biological machinery in a cell-free system, that is, without the use of living cells. The CFPS environment is not constrained within a cell wall or limited by conditions necessary to maintain cell viability, and enables the rapid production of any desired protein from a nucleic acid template, usually plasmid DNA or RNA from an in-vitro transcription. CFPS has been known for decades, and many commercial systems are available. Cell-free protein synthesis encompasses systems based on crude lysate (Cold Spring Harb Perspect Biol. 2016 December; 8(12): a023853) and systems based on reconstituted, purified molecular reagents, such as the PURE system for protein production (Methods Mol Biol. 2014; 1118: 275-284). CFPS requires significant concentrations of biomacromolecules, including DNA, RNA, proteins, polysaccharides, molecular crowding agents, and more (Febs Letters 2013, 2, 58, 261-268).

Split detectors such as split green fluorescent protein (GFP) systems are known for use in protein expression. A protein of interest having a GFP subcomponent can be detected by complementing with a detector species having the remainder of the GFP. Cabantous and Waldo describe such a system (In vivo and in vitro protein solubility assays using split GFP (NATURE METHODS|VOL.3 NO.10|OCTOBER 2006|845). The system described relies on expression in cells, from which the proteins of interest are lysed and then exposed to the detector in order to measure the level of expression.

WO2022/038353 describes cell-free expression of a protein having a GFP tag in the presence of a GFP detector species to measure signal as expression progresses.

Many proteins are expressed containing endogenous solubility factors. However factors such as for example maltose binding protein (MBP), Small Ubiquitin-like Modifier (SUMO), Glutathione S-transferase (GST) or thioredoxin (TRX) have a substantial size and may have undesirable effects on the proteins of interest to which they are attached. Additionally expression of large solubility elements uses the reagent resources during expression. Such sequences also take time to fold before acting at solubility enhancers, hence synthesising proteins of interest with solubility enhancing sequences attached thereto is far from ideal. The applicants herein wish to produce proteins without requiring attached solubility factors.

To date protein purification and analysis typically requires complex analysis techniques involving electrophoresis or requiring purified proteins. The inventors herein have developed simplified protein analysis methods allowing multiple ways of characterising expressed proteins in their crude form.

WO2007112677 and CN112646826 disclose expression with fusion proteins such as Trx. Trx is a protein commonly found in yeast, bacteria, animals, and plants, and this protein is also an endogenous protein of commonly used hPTH(1-34) expression hosts such as yeast or Escherichia coli. This protein can regulate the balance of protein folding and aggregation process in the cell. Trx can interact with many proteins and enhance the solubility of fusion proteins, thereby reducing the formation of inclusion bodies. The fusion protein has the TRX attached to the protein of interest.

US2006099710 discloses expression of a MBP fusion protein where the MBP improved solubility of the expressed protein The vector contains a sequence expressing a protease cleavage site (TMV protease). The expression occurs and the MBP is cleaved. The MBP fusion aids solubility of the expressed protein by being attached to the sequence of the protein of interest.

KR101828672 describes an introduction vector having a DNA sequence encoding 165 amino acids of IFNα2b which was codon-optimized and synthesized. Seven tags (His6, Trx, GST, hPDlb‘a’, MBP, hPDl and NusA) were fused to the N-terminus of IFNα2b. A Tobacco etch virus recognition site (TEVrs; ENLYFQ↓G (SEQ ID NO: 1)) for removal of tagged proteins was placed between the tag and IFNα2b of each construct. As above the solubility tags are directly attached to the proteins of interest in a contiguous sequence.

WO2018076008 again discloses fusion proteins with solubility tags attached to proteins of interest.

US2022275027 again discloses fusion proteins with signal peptides attached to proteins of interest.

CN108117599 again discloses fusion proteins comprising a chaperone and a toxin polypeptide Ssm6a (also referred to as “the fusion protein of the invention”), wherein the molecular chaperone promotes correct folding of the disulfide bonds in the toxin polypeptide.

WO2022038353 describes the use of split fluorescent proteins for detecting protein expression, but is not used as a measure for aggregation.

WO2014132053 describes microdroplet assays for protein aggregation, but is not used as an assay for protein expression.

One of the current challenges for cell-free protein synthesis is to increase the soluble yield of the expressed proteins and to avoid aggregation or insolubility.

### SUMMARY OF THE INVENTION

The invention relates to methods for determining the degree of aggregation of a protein. The methods relies of expressing a expressing a protein of interest having a binding tag that upon binding to a detector moiety generates a detectable signal; contacting with a detector moiety to generate a detectable signal upon binding to the tag; and measuring the homogeneity of the signal generated upon binding of the detector moiety to the binding tag to determine the level of aggregation of the protein of interest.

The generated signal may be fluorescent. The fluorescence measurement can be performed using techniques for measuring fluorescence through small path lengths of fluid (for example less than 1 mm or less than 200 μm). For example the measurement may be performed using flow cytometry, in thin films or in capillary flow systems. The measurement may be performed in low path length cuvettes. The low path length cuvettes are typically less than 1 mm path length, and may be 0.05 mm to 1 mm in path length. The measurement may be performed on a nanodrop type fluorimeter, such as for example the Qubit Fluorometric Quantification System (Thermo Fisher).

Flow cytometry is a powerful technique used in biology and medicine to analyze the physical and chemical characteristics of particles, as they flow in a fluid stream through a beam of light. As each particle crosses the beam, it scatters light and emits fluorescence, providing valuable information about its size, complexity, and molecular composition. This allows investigation of the particle's properties, such as size, granularity, and fluorescence intensity, at the single-particle level. Flow cytometry provides quantitative and qualitative information about heterogeneous populations, and can rapidly analyze thousands of particles per second with high precision.

The signal can be measured in for example droplet sprays or in capillary systems such as systems involving microfluidic chips consisting of microfabricated channels and chambers on a small substrate, typically made of glass, silicon, or polymers like PDMS (polydimethylsiloxane). The capillary systems may be microflow type systems where the analysis is performed in a flow of material past a detector.

Fluorescence can be effectively measured in thin films using various spectroscopic and microscopic techniques. Thin films are layers of material with a thickness typically on the order of micrometers (1-500 μm). Expression in thin films allows localised measurements of aggregation. The thin films may be produced by spreading material onto a surface. Aqueous liquid spread only surfaces having high surface energy. Liquids may be spread on a surface by increasing the surface energy of the surface. For example the film may be produced using electrowetting in order to reduce the thickness of a droplet. Alternatively the surface may be hydrophilic in order to have a low contact angle when liquid is applied. Thus expression may be performed in solution and droplets applied to a hydrophilic surface in order to measure signal uniformity.

Fluorescence spatial correlation analysis involves quantifying the spatial relationships between fluorescence intensity values at different locations within a sample. This analysis provides insights into the spatial organization and homogeneity of fluorescent molecules or structures within the sample.

Alternatively to measuring fluorescence, techniques such as surface plasmon resonance (SPR) may be used. In such cases the binding tag expressed with the protein acts to immobilise the protein. The presence of monomers or multimers can be detected directly using the resonance signal from the captured material. Additional tags may be used to attach extra mass and increase the signal from the immobilised material if needed. The use of binding ligands may be studied in order to increase or decrease aggregation. SPR is a powerful technique for studying biomolecular interactions in real time and without the need for labeling. When proteins aggregate, they often undergo changes in conformation and size, which can affect their interaction with surfaces. SPR measures changes in the refractive index near a sensor surface caused by the binding of molecules to that surface. When protein aggregates bind to the sensor surface, they can cause detectable changes in the SPR signal, such as alterations in the angle or intensity of reflected light.

The invention relates to a method for determining the degree of aggregation of a protein in one or more droplets on a digital microfluidic device, where the degree of aggregation is measured using an optical method. On one aspect, the invention relates to determining the degree of aggregation of an expressed protein in a cell-free system comprising expressing a protein of interest (POI) in droplets on a digital microfluidic device, where the expressed POI is measured using a detectable signal and measuring the levels of insoluble POI. The level of aggregation can be used to determine whether a protein has expressed as soluble or insoluble. Even if a protein expresses solubly, it doesn't mean that the protein will remain soluble. By looking at the aggregation status it informs regarding the “aggregation propensity” or “stability” of the initially soluble protein. Soluble protein may be further purified, whereas proteins with a high degree of aggregation are unlikely to be able to be purified or to be active.

Fluorescence intensity may be measured using a suitable multi-pixel camera. Each area covered by the liquid sample occupies multiple camera pixels. Thus the homogeneity of the signal can be calculated by measuring the intensity of the signal for each pixel in the sample image and compared for example using a standard deviation. Cumulative Intensity is the sum of the pixel values in the droplet area. Dependent on image bit depth, for 16-bit images a pixel is between 0 and 65535 and for 8-bit between 0 and 255. Raw Intensity measures the average pixel intensity in the droplet area and is dependent on image bit depth. Std. Dev. (Intensity) is the standard deviation of the intensities in the droplet area. Cluster Score is a score which indicates how likely a droplet is to have clusters. Anything above a threshold may have a further confirmation analysis performed to detect clusters. Intensity (Non-Cluster) is the average intensity of an droplet when cluster regions are excluded.

Expression and purification metrics may be determined and ranked using a score for the ratio of sample homogeneity. The score may be calculated for example using a measure of total intensity divided by the intensity from clusters or aggregates. The score may give a measure of smoothness across the measurement volume. A high level of signal from clusters gives a low score and a lack of smoothness (high heterogeneity). A high score or level of smoothness may be obtained where the signal intensity is uniform and few or no clusters are seen (low level of heterogeneity). The ‘score’ value may be for example the droplet intensity with clusters excluded divided by a clustering measure. Excluding the clusters means that this intensity is reflective of the soluble protein within the droplet. Clusters are assumed to contain protein which is ‘inaccessible’ in some way, and samples having high levels of aggregation are less suitable for further purification or analysis. A clustering measure can be determined by the ratio of intensity when clusters included and excluded. This score should be 1 when there is no clustering (and thus nothing excluded), and increases when clusters are brighter or have a larger area as more signal is subtracted. The effect of cluster subtraction may be more pronounced if the non-cluster intensity from the droplet is small, i.e. little overall protein expression is observed.

The analysis of images typically plots intensity across the sample or droplet. Bright spots localised within the sample are indicative of aggregation. The method may use a binary threshold where all image values are set to either 0 (below threshold) or 1 (above threshold). Values above the threshold may be subject to a more thorough analysis. Another method would be to find peaks in the region of interest, where a peak is a local maxima (i.e. greater than its nearest neighbours). A bright spot is a for example a peak with a magnitude >x (where x is defined based on the average across the sample). A common image processing library is opencv in python, which has functions specifically designed to find brightspots, peaks etc. OpenCV (Open Source Computer Vision Library: opencv dot org) is an open-source library that includes several hundreds of computer vision algorithms, and which may be used.

Aggregation measurements may rely on molecular sizing techniques. The fluorescence may be measured using techniques such as Flow Induced Dispersion Analysis (FIDA). FIDA involves measuring fluorescence of particles in a laminar flow and analysing their dispersion over time, which allows for calculation of the hydrodynamic radius of a particle of interest. Larger molecules disperse more slowly. Measurements are typically performed in a capillary.

The fluorescence may be measured by blending the fluorescently labelled proteins with a blank buffer solution and monitoring the rate of diffusion. Larger aggregates diffuse more slowly across the combined liquid volume. Such a method is disclosed in WO2023/079308, which is incorporated herein by reference.

The protein for screening may be expressed on device or may be placed on the device from an external sample, either sourced as a pure protein or via in-tube (off device) expression. The protein may be used in a stability screen, for example to identify buffers and surfactants in which the protein is more or less prone to aggregation. For example the protein sample may be screened against a set of buffers and surfactants (i.e. 24 buffers vs 8 different surfactants) to identify a reagent that the protein is more stable in. In such a case the method is selecting against aggregates in the search for an optimised buffer in a highly parallelize buffer screen. The rate of aggregate formation may be used estimate protein stability in the varying conditions.

The degree of aggregation can be measured directly, for example using light scattering, or may be seen using visible labels. The detection may be seen as optically detectable aggregates which cause light scatter, or may give rise to aggregates which can be detected by labelling, for example using fluorescence. The light scattering may be measured at an angle different from the incident light. The POI may have a binding sequence which binds to a detector moiety. The detector moiety can be added after the expression by combining additional droplets.

The degree of aggregation can be measured by counting the number of aggregates, the area of the aggregates, the intensity of the aggregates or by using a measurement of turbidity or dispersion. The degree of aggregation may be zero, indicating the droplet contents are homogeneous and remain in solution. In such instances the optical or fluorescence signal is evenly distributed across the whole droplet with no clustering or clumping. Alternatively the aggregation may be complete such that only aggregates are detected as clusters, and the remaining droplet gives no signal. On a scale between the two extremes, the signal may be partially aggregated and partially dispersed across the whole droplet.

The POI may be expressed with or contain a binding sequence which binds to a detector moiety. In which case a further droplet containing a detector moiety may be added to the droplet containing the POI to create an detectable signal in the combined droplet.

The binding sequence may contain four or more amino acids. The binding sequence may contain 4-30 amino acids.

The detector moiety may be a protein. The detector moiety may comprise a component of a fluorescent protein. The fluorescent protein may contain a further solubility enhancer, for example selected from:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation factor IF2 | IF2-domain 1 |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension sequence | NEXT |

The tag may be for example a GFP₁₁ tag. Upon expression of the POI, once in the presence of the added GFP₁₋₁₀, the fluorescent complex GFP₁₋₁₁ is formed.

The GFP₁₁ is of sufficient length to bind to GFP₁₋₁₀ to create a stable binding and a fluorescent signal from the GFP₁₋₁₁. The GFP₁₁ peptide amino sequence tag may be selected from:

(SEQ ID NO: 2)
KRDHMVLLEFVTAAGITGT
(SEQ ID NO: 3)
KRDHMVLHEFVTAAGITGT
(SEQ ID NO: 4)
KRDHMVLHESVNAAGIT
(SEQ ID NO: 5)
RDHMVLHEYVNAAGIT
(SEQ ID NO: 6)
GDAVQIQEHAVAKYFTV
(SEQ ID NO: 7)
GDTVQLQEHAVAKYFTV
(SEQ ID NO: 8)
GETIQLQEHAVAKYFTE.

If desired the tag may be cleaved from the POI in order to remove the tag/detector. The cleavage may be performed for example using a protease of a metal cation.

Expression of the expressed protein may be determined, for example using an optical measurement without purification from the expression system. The expression and/or stability may be determined at a fixed time or temperature. For example exposing the non-purified protein to a set temperature and analysing said sample for the presence of remaining soluble POI over time or exposing the non-purified protein to a range of temperatures and analysing said sample for the presence of remaining soluble POI.

The stability may be determined by expressing a protein of interest (POI), splitting non-purified expression into multiple portions, exposing at least one portion of the non-purified protein to an elevated temperature and analysing said heated sample for the presence of remaining soluble POI when compared to an unheated portion of the sample.

The stability may be determined by taking a protein of interest (POI), splitting the protein sample multiple portions, exposing at least one portion of the protein to an elevated temperature and analysing said heated sample for the presence of remaining soluble POI when compared to an unheated portion of the sample.

The stability may be determined by taking a protein of interest (POI), splitting the protein sample multiple portions, merging additives with the protein droplets to generate varying chemical compositions such as varying buffers and surfactants and analysing said varying compositions for the presence of remaining soluble POI when compared to an untreated portion of the sample.

Once an amino acid sequence is produced by expression, activity depends on the correct folding. Solubility and activity are dependent on the stability of the expressed protein. An incorrectly folded protein may be less stable than a correctly folded protein. Thus the stability of the expressed protein under test conditions is an important measure of the presence of the correctly folded protein. Following protein expression, a rapid system of characterisation of the resultant proteins can be used in order to determine the stability of the expressed material without having to purify the material.

Protein stability indicates the ability of a protein to retain correct folding and/or to not aggregate over time. Stability depends on the conditions, e.g. protein concentration, pH, buffer composition or temperature. Each set of variable conditions can be determined, for example thermal stability, aggregation propensity, pH stability etc. Measuring the stability of expressed proteins is important when determining activity in further assays in order to ensure the measured properties are of the desired folded proteins, rather than oligomers, aggregates or other mis-folded structures. Assays for protein stability may include a change in solubility, aggregation, thermal stability upon heat changes or the use of known binding ligands.

The method may be used to characterise an initial soluble yield of the expressed protein and the stability of the expressed protein. The method may identify the optimal conditions for both expression and stability of an expressed protein.

The aggregation may be measured directly or by using addition of reagents which detect aggregation. For example using thioflavins. Thioflavins are fluorescent dyes that are available as at least two compounds, namely Thioflavin T and Thioflavin S. Both are used for histology staining and biophysical studies of protein aggregation, for example to investigate amyloid formation. Thioflavin T has been used in research into Alzheimer's disease and other neurodegenerative diseases.

The stability may be determined using dynamic light scattering (DLS) of aggregated protein clusters. The stability may be determined using differential scanning fluorimetry (DSF). The stability may be determined by taking time points at a fixed temperature as an isothermal stability test. Alternatively the stability may be measured by heating to different temperatures for a fixed time. The stability may be measured by heating the expressed protein to for example greater than 40° C. or greater than 50° C.

The stability may be measured by analysing the remaining soluble fraction or the level of aggregation remaining after the heating step.

The stability may be determined over a period of time and compared to a starting measurement. The method may involve expressing a protein of interest (POI), monitoring the amount of soluble expression, exposing the non-purified protein to a set temperature and analysing said sample for the presence of remaining soluble POI over time.

The method may involve expressing a protein of interest (POI), monitoring the amount of soluble expression, exposing the non-purified protein to a range of temperatures and analysing said sample for the presence of remaining soluble POI.

The method may involve expressing a protein of interest (POI), splitting non-purified expression into multiple portions, exposing at least one portion of the non-purified protein to an elevated temperature and analysing said heated sample for the presence of remaining soluble POI when compared to an unheated portion of the sample. The method may involve expressing a protein of interest (POI) in multiple separate volumes of liquid, exposing at least one volume of the non-purified protein to an elevated temperature and analysing said heated volume for the presence of remaining soluble POI when compared to an unheated volume.

The expression may be performed using cell-free protein synthesis reagents derived from whole cell extracts. The expression may be performed using cell-free protein synthesis reagents derived from reconstituted systems comprising assembled components for transcription and translation in a system of purified recombinant elements (PURE).

Any method described herein can be performed in for example microtitre plates or microcentrifuge tubes. Expressed protein can be placed in device for a high throughput stability screen. Any method may be performed on a microfluidic or digital microfluidic device. The digital microfluidic device may comprise an oil-filled or humidified gaseous environment, wherein the humidified gaseous environment is achieved by enclosing or sealing the digital microfluidic device and providing on-board reagent reservoirs.

Disclosed is a method for expressing proteins in droplets on a digital microfluidic device having a two-dimensional array of planar microelectrodes wherein the proteins have a GFP₁₁ peptide amino sequence tag, wherein a portion of the droplets contain GFP₁₋₁₀ during the expression process and a further portion have GFP₁₋₁₀ added after expression and comparing the level of GFP₁₋₁₁ signal from the droplets.

The expression comparison can identify the optimal conditions for expression of the desired protein in its most soluble and stable form.

### FIGURES

FIG. 1 shows a protein of interest with a peptide tag (detection tag) in the presence of a protein that binds to the peptide tag (detector protein). In the case that the detection tag and detector protein generate a detectable signal upon binding, then the quantity of the protein of interest can be measured. In addition/alternatively, if the detector protein is a solubilising factor, then it may increase the solubility of the protein of interest upon binding to the peptide tag. The solubilising power of such a binding system can be superior to the solubilising power of a fusion protein solubility factor.

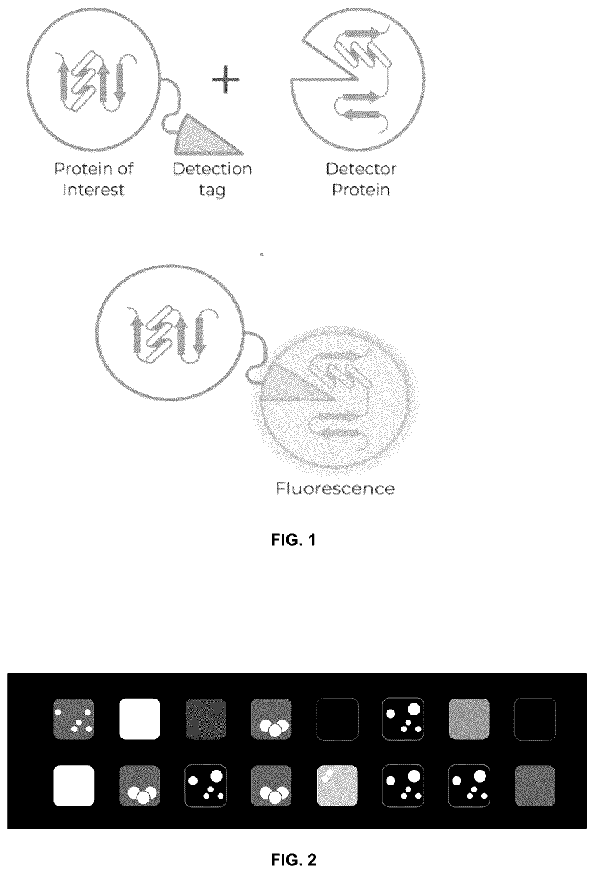

FIG. 2 shows a cartoon representation of protein expression of different proteins having varying soluble yields and levels of aggregation. Some conditions give no expression (dark). Some conditions give a high level of soluble protein (uniform intensity white squares). Some conditions give protein aggregates, meaning the expressed proteins are insoluble and clump together (white spots in dark background). Some give a mix of soluble and aggregated proteins (white spots in a grey background). Thus the level of soluble expression and level of aggregation can be determined by adding a detector species to the expressed proteins.

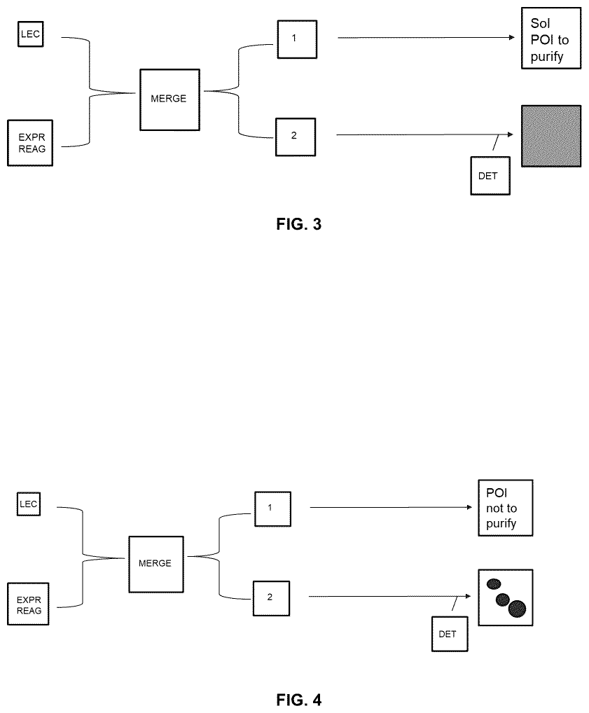

FIG. 3 shows a schematic workflow for protein expression. DNA linear expression constructs (LEC) can be mixed with expression reagents and merged. The mixed reaction can be split into multiple aliquots and left to undergo protein expression. A soluble detector species (DET) is added to one drop after expression has occurred. Where the expressed protein is soluble and the degree of aggregation is low, the signal is uniform, showing the protein is solubly expressed.

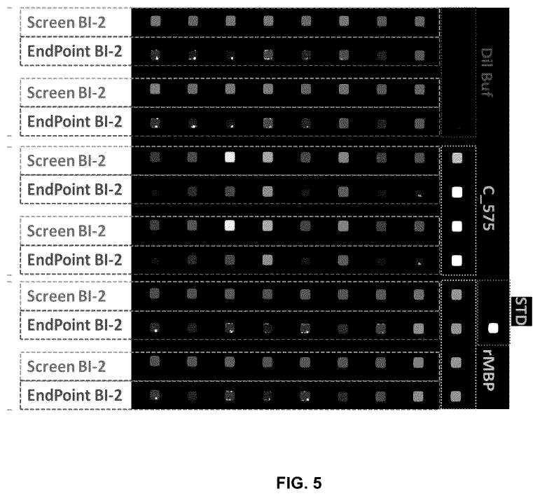

FIG. 4 shows a schematic workflow for protein expression. DNA linear expression constructs (LEC) can be mixed with expression reagents and merged. The mixed reaction can be split into multiple aliquots and left to undergo protein expression. A soluble detector species (DET) is added to one drop after expression has occurred. Where the expressed protein is aggregated, the detector gives a non-uniform signal, indicating the expressed protein is to some degree insoluble or aggregated. The insoluble or aggregated POI is not likely to be purified from the droplets.

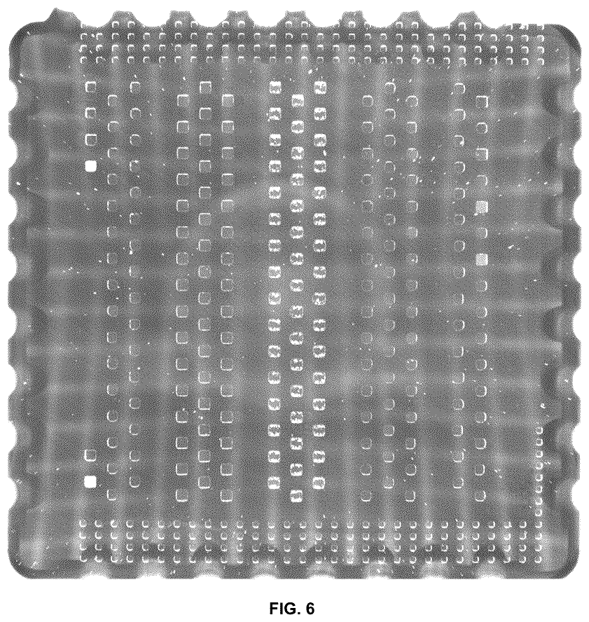

FIG. 5 shows the experimental result from 24 different proteins expressed in a reconstituted cell-free protein synthesis system in droplets on an electrowetting on dielectric (EWoD) device. Each construct contains a GFP₁₁ tag. In the rows marked Screen, the GFP₁₋₁₀ detector species is present from the start of expression. The rows marked Endpoint shows the fluorescence signal from 10 hours expression in the absence of GFP₁₋₁₀ detector species followed by 5 hours complementation with the GFP₁₋₁₀ detector species. This experiment showed significant differences between expression/complementation in Screen BioInk compared to Endpoint detection. The detected protein clusters formed after expression only with endpoint detection mean that the protein aggregated after expression, thereby lowering the soluble yield. It is evident from the image the presence of aggregates for several constructs, indicating the likely presence of protein aggregates. The level of aggregation enables identification of conditions which are worthy of further testing, and identification of conditions having high level of aggregated protein from which further purification is unlikely to give material.

FIGS. 6-8 show expression results from two proteins expressed in a reconstituted cell-free protein synthesis system in droplets on an electrowetting on dielectric (EWoD) device. 8 different cell-free expression blends are tested for each of the proteins. FIG. 6 shows a white light image after 10 h expression. FIG. 7 shows a white light image after addition of detector protein (further 5 h). FIG. 8 shows a fluorescent (blue light) image after addition of detector protein. For a soluble protein (DHFR) (left side of array) the droplets appear uniform in the three images as the expressed protein does not appear as clumps, no clumping or aggregation can be seen in any of the 3 images. In the white light images the droplets appear with varying levels of turbidity depending on the level of expression. In the central 3 columns of the image after the expression (FIG. 6) clusters of protein (P17_IFN) can be detected in the optical image. The clusters remains detectable in the white light images after the detector protein has been added (FIG. 7). The fluorescence images (FIG. 8) show clusters of fluorescence within the droplets, indicating the presence of protein aggregates. Droplets having a high level of turbidity in the white light images also have a high level of signal from bond detector protein, indicating that protein can be detected either using white light or fluorescence measurements.

### DETAILED DESCRIPTION OF THE INVENTION

Applicants are able to visualise protein aggregates directly in droplets on a microfluidic device. Such visualisation methods allow protein stability to be seen directly using an optical readout. Applicants have appreciated that the level of aggregation following protein expression is an important metric in determining whether a protein can be subsequently purified. A high level of protein aggregation indicates a lack of soluble protein for purification. In such cases the protein is expressed, but aggregates or mis-folds after expression, in contrast to a protein which fails to express the correct sequence. Disclosed herein is a method for determining the degree of aggregation of an expressed protein in a cell-free system comprising expressing a protein of interest (POI) in droplets on a digital microfluidic device, where the expressed POI is measured by an optically detectable signal and measuring the levels of insoluble POI.

Further disclosed is a method for protein synthesis comprising expressing a protein of interest (POI) and measuring the stability of the expressed protein using an optical measurement without purification from the expression system.

When expressing proteins, various parameters are important for determining successful expression, both in terms of yield and function. One of the factors is the soluble yield of expressed proteins, as many pertains express in forms that are insoluble or become inactive through a lack of stability.

The assays for soluble yield described herein involve expression and detection of the expressed proteins to measure the degree of aggregation. The protein of interest (POI) may have a binding tag which binds to a detector to generate a signal. The detector may have a further solubility factor.

The method may be used as part of a process to determine whether an expressed protein is suitable for subsequent purification testing. For example the method may involve expressing a protein of interest (POI) in a first reagent volume and splitting the reagent volume into multiple aliquots. The protein of interest may have a binding tag which complements a detector species and becomes fluorescent. The detector species is added to one or more of the aliquots and the signal recorded. The uniformity of the signal in the droplet determines the degree of aggregation. Where the signal is homogenous across the droplet, the POI is soluble, with little or no aggregation. Where the signal is non-homogenous across the droplet, seen in the form of clumps, the protein is aggregated and insoluble. Thus the level of overall signal indicates the level of expression and the signal uniformity determines aggregation. Thus the level of expression, and the level of soluble expression can be determined from looking at the amount of, and uniformity of the signal within the droplet. The degree of aggregation may be measured by using a measurement of uniformity, counting the number of aggregates, the area of the aggregates, the intensity of the aggregates or by using a measurement of dispersion.

Proteins may give rise to aggregation if the proteins are unstable or poorly soluble. Protein aggregates give rise to a lack of homogeneous signal in white light optical images or from adding the detector. Thus POIs having a high soluble yield give a uniform level of signal, proteins having a low soluble yield may have either a low or high level of signal, but the signal aggregates and is not homogenous throughout the reaction volume as the proteins are aggregated. Proteins which are aggregated are likely to be less desirable for subsequent assays or purification.

Further assays for protein stability may include
Solubility
Aggregation (over time)
Thermal Stability

Protein thermal stability does not necessarily predict aggregation rate at low temperature (and vice versa). For example, the melting temperature (Tₘ) of a monoclonal antibody does not necessarily correlate to the aggregation rate of said monoclonal antibody at room temperature (Mol. Pharmaceutics 2016, 13, 307-319). At high temperatures, the aggregation rate is dependent on the unfolded state whereas at low temperature, the aggregation rate is dependent on the native state. Thus denaturing a protein may or may not give an accurate measure of stability for the protein in its native state.

Providing a protein aggregation metric over time would be highly useful and present additional information in addition to simply a measurement of the expressed yield. Producing stable protein formulations is key to the development and manufacturing of proteins. The robustness of the formulations against external stress factors is crucial as proteins may be exposed to various types of stress such as temperature, pH, salts, mechanical stress, surface interaction or oxidation. Protein aggregation is a common issue faced during expression and purification. Therefore, the prediction or control of protein aggregation during the production of a protein of interest is much needed. Described herein is a high throughput assay for protein aggregation/stability which measures the protein stability using the directly expressed material.

Protein purification can be challenging and time consuming, often resulting in a significant loss of material. Therefore it is desirable to measure protein stability directly after expression without having to purify the proteins from the proteins used in expression. Measurement in crude cell lysates or directly in cell-free expression systems are possible using optical based analysis techniques, as these can tolerate the presence of other proteins which are stable to the conditions being measured or are not otherwise detectable.

Disclosed is a method for protein synthesis comprising expressing a protein, wherein the expressed protein contains a sub-component of a fluorescent protein, the method comprising monitoring stability of the protein over time by creating the fluorescent protein in different batches of material at different times.

The method avoids the need for complex purification steps or gel based separations. A fluorescence based assay can be used to measure the amount of expressed protein (soluble yield) in conjunction measuring protein stability. Thus both yield and stability can be determined after expression without needing to run gels or purify material.

Optical based assays for protein stability may include a change in solubility, aggregation, thermal stability upon heat changes or the use of known binding ligands.

A binding ligand assay may for example use a known binding ligand to increase or decrease the stability of the protein. Thus the presence and absence of the ligand can be compared directly and the effect on stability monitored. The binding ligand may be for example a chaperone protein or co-factor. The chaperone may be co-expressed during the expression step, may be present in the expression mixture or may be added after the expression is complete. The ligand can be added to the expressed protein after expression, for example by adding an extra droplet on a digital microfluidic device. The ligand may be added simultaneously with a detector that measures the expressed protein.

The optical measurement may be for example light scattering caused by aggregates of the expressed protein. When a protein aggregates and clusters together, the increased size of the protein aggregates cause light to scatter, which can be detected.

The measure may use a thermal shift assay. A thermal shift assay (TSA) measures changes in the thermal denaturation temperature and hence stability of a protein under varying conditions such as variations in drug concentration, buffer pH or ionic strength, redox potential, or sequence mutation. The most common method for measuring protein thermal shifts is differential scanning fluorimetry (DSF), which utilizes specialized fluorogenic dyes.

U.S. Pat. No. 9,528,996 describes methods for determining ligand binding to a target protein using a cellular thermal shift assay. The technique described, (CETSA®), is directed to a method of determining whether a non-purified sample contains a target protein bound to a ligand of interest. The method comprises the steps of: a) exposing said non-purified sample to a temperature which is capable of causing or enhancing precipitation of the unbound target protein to a greater extent than it is capable of causing or enhancing precipitation of the target protein bound to said ligand; b) processing the product of step a) in order to separate soluble from insoluble protein; and c) analyzing either or both the soluble and insoluble protein fractions of step b) for the presence of target protein. Particularly, the invention may be used to determine whether drugs can bind to their protein targets in samples derived from patients to ascertain whether a certain drug can be used in a therapy for that patient.

The CEllular Thermal Shift Assay (CETSA®) leverages the insight that melt curves of proteins can also be measured in intact cells and that proteins unfold and aggregate at individual and specific temperatures. The very basis for thermal shift assays, cellular or not, is that the thermal stability of proteins can change if their environment is altered. In its simplest form, CETSA® is carried out on a single protein basis, assessing target engagement qualitatively by incubating and heat shocking cells in the presence and absence of the studied ligand compound. Quantifying the amount of protein that remains soluble after heat shock and plotting this to a range of different temperatures gives the CETSA® melt curve of the protein. If the binding compound has bound to the target protein the latter becomes more or less resistant to heat, causing a shift in the melt curve (also known as a thermal shift).

Target engagement can also be quantified by exposing the cells to different concentrations of the compound and comparing the extent of stabilization of the target protein at specific temperatures. Such concentration-response experiments allow CETSA® specific potencies of target engagement to be determined. These potencies incorporate more than just the protein-ligand affinity, they also include factors such as membrane permeability, cellular activation or degradation, accessibility of the protein binding site. However CETSA requires the use of a binding ligand, and also relies on the generation of melt curves at varying temperatures.

Where the expressed protein has a known binding ligand, disclosed herein is the use of CETSA® to determine the stability of expressed proteins directly without needing purification of the expression components. The protein can be expressed to have a binding site for a known ligand, thereby introducing a known binding site.

Differential Scanning Fluorimetry (DSF) measures protein unfolding by monitory changes in fluorescence as a function of temperature. Conventional DSF uses a hydrophobic fluorescent dye that binds to proteins as they unfold. NanoDSF measures changes in intrinsic protein fluorescence as proteins unfold.

The fluorescence measurement can act as a real time measure of soluble protein expression. As the tag sequence is produced, the presence of the remaining fluorescent protein as a detector species then allows real-time measurement of the tag sequence and the amount of soluble protein. Alternatively the fluorescence can be measured by adding the detector species after expression to measure insoluble protein. The fluorescence can be retained and monitored during the stability assay. Alternatively further detector protein can be added after the stability step to determine the remaining protein level. Both yield of synthesis and stability of expressed protein can thus be determined. For example GFP₁₁ can be attached to the expressed protein and GFP₁₋₁₀ used as the detector species.

This screening workflow enables users to rapidly screen different expression systems in the form cell-free lysates, or reconstituted systems. Having identified an optimal expression system, soluble yield and stability can be measured directly without having to separate a protein of interest from a complex mixture containing other proteins, nucleic acids, and other cellular components.

The protein can be expressed with a binding tag. The binding moiety can be a region of amino acid/peptide sequence. The affinity binding site can be a region of amino acid/peptide sequences specific to a particular antibody. The tag can be attached to the N or C terminus. For example the binding moiety can be selected from the list of exemplary peptide affinity binding sites below:

Alfa-tag
(SEQ ID NO: 9)
(SRLEEELRRRLTE)
Avi-tag
(SEQ ID NO: 10)
(GLNDIFEAQKIEWHE)
C-tag
(SEQ ID NO: 11)
(EPEA)
Calmodulin-tag
(SEQ ID NO: 12)
(KRRWKKNFIAVSAANRFKKISSSGAL)
Dogtag
(SEQ ID NO: 13)
(DIPATYEFTDGKHYITNEPIPPK)
E-tag
(SEQ ID NO: 14)
(GAPVPYPDPLEPR)
FLAG
(SEQ ID NO: 15)
(DYKDDDDK)
G4T
(SEQ ID NO: 16)
(EELLSKNYHLENEVARLKK)
HA
(SEQ ID NO: 17)
(YPYDVPDYA)
His
(SEQ ID NO: 18)
(HHHHHH)
Isopeptag
(SEQ ID NO: 19)
(TDKDMTITFTNKKDAE)
lanthanide binding tag
(LBT)
(SEQ ID NO: 20)
(FIDTNNDGWIEGDELLLEEG)
Myc
(SEQ ID NO: 21)
(EQKLISEEDL)
NE-Tag
(SEQ ID NO: 22)
(TKENPRSNQEESYDDNES)
Poly Glutamate-tag
(SEQ ID NO: 23)
(EEEEEEE)
Poly Arginine-tag
(SEQ ID NO: 24)
(RRRRRRR)
Rho1D4-tag
(SEQ ID NO: 25)
(TETSQVAPA)
SBP-tag
(SEQ ID NO: 26)
(MDEKTTGWRGGHVVEGLAGELEQLRARLEHHPQGQREP)
Sdytag
(SEQ ID NO: 27)
(DPIVMIDNDKPIT)
SH3
(SEQ ID NO: 28)
(STVPVAPPRRRRG)
SNAC
(SEQ ID NO: 29)
(GSHHW)
Snooptag
(SEQ ID NO: 30)
(KLGDIEFIKVNK)
Softag 1
(SEQ ID NO: 31)
(SLAELLNAGLGGS)
Softag 3
(SEQ ID NO: 32)
(TQDPSRVG)
Spot-tag
(SEQ ID NO: 33)
(PDRVRAVSHWSS)
Spytag
(SEQ ID NO: 34)
(AHIVMVDAYKPTK)
S-tag
(SEQ ID NO: 35)
(KETAAAKFERQHMDS)
Strep-tag
(SEQ ID NO: 36)
(AWAHPQPGG)
(SEQ ID NO: 37)
(AWRHPQFGG)
Strep-tag II
(SEQ ID NO: 38)
(WSHPQFEK)
T7tag
(SEQ ID NO: 39)
(MASMTGGQQMG)
TC-tag
(SEQ ID NO: 40)
(EVHTNQDPLD)
Ty-tag
(SEQ ID NO: 41)
(CCPGCC)
VSV-tag
(SEQ ID NO: 42)
(YTDIEMNRLGK)
Xpress-tag
(SEQ ID NO: 43)
(DLYDDDDK)

The binding moiety tag can be a sub-component of a fluorescent protein. Thus the fully assembled protein becomes fluorescent. For example if the detector protein contains GFP₁₋₁₀ and the expressed protein tag contains a GFP₁₁ peptide, complementation forms fluorescent GFP, allowing simultaneous monitoring and stability evaluation. The expressed material can be monitored over time or conditions by monitoring changes in the level of aggregated material generating a fluorescent signal.

The binding moiety may include a small molecule affinity tag such as biotin. The binding moiety may include a particular sequence of nucleic acids.

The process can be performed in droplets, which can be manipulated by electrokinesis in order to effect and improve protein expression and analysis. The droplet can be moved using any means of electrokinesis. The droplet can be moved using electrowetting on dielectric (EWoD). The electrical signal on the EWoD or optical EWoD device can be delivered through segmented electrodes, active-matrix thin-film transistors, or digital micromirrors.

The cell-free expression of peptides or proteins can use a cell lysate having the reagents to enable protein expression or a reconstituted system. Common components of a cell-free reaction include an energy source, a supply of amino acids, cofactors such as magnesium, and the relevant enzymes. A cell extract is obtained by lysing the cells of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery.

In order to optimise expression, the expression system can be supplemented with additional components, including purified enzymes. The additional components may include salts, co-factors, buffers, surfactants, chaperones or additional protein components. The additional protein components may be selected from for example chaperones, glycosylating enzymes, proteases, redox active enzymes, phosphorylases and kinases.

The expression composition may be assembled on the device from mixing a variety of droplets in order to screen a variety of compositions in parallel.

By way of example, the screening reagents may include,
Chaperone mix (e.g., PUREfrex GroE mix)
Kinase 1 (e.g., NEB CK2)
Kinase 2 (e.g., NEB PKA)
Protease 1 (e.g., NEB TEV)
Protease 2 (e.g., Merck HRV 3C)
Common metal ions cocktail
Common co-factors cocktail

The compositions can be blended by the user and the level of expression of the protein of interest monitored in each of the blended conditions.

Any particular nucleic acid template can be expressed using the system described herein. Three types of nucleic acid templates used in cell-free protein synthesis (CFPS) include plasmids, linear expression constructs (LECs), and mRNA. Plasmids are circular templates, which can be produced either in cells or synthetically. LECs can be made via PCR. mRNA can be produced through in-vitro transcription systems. The methods can use a single nucleic acid template per droplet. The methods can use multiple nucleic acid templates per drop. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

The cell-free extract having the components for protein expression includes everything required for protein expression apart from the nucleic acid template. Thus the term includes all the relevant ribosomes, enzymes, initiation factors, nucleotide monomers, amino acid monomers, metal ions and energy sources. Once the nucleic acid template is added, protein expression is initiated without further reagents being required.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied. The PURE system is composed of all the enzymes that are involved in transcription and translation, as well as highly purified 70S ribosomes. The protein synthesis reaction of the PURE system lacks proteases and ribonucleases, which are often present as undesired molecules in cell extracts.

The use of a population of droplets having different components allows the rapid screening of a variety of variable factors to identify optimal conditions for expression of a desired proteins. The protein can be contained with a sequence having other amino acid domains, for example solubility factors or binding tags.

Protein solubility sequences may be attached to the detector sequences. For example the GFP₁₋₁₀ may be attached to further elements to improve solubility. The solubility enhancing sequence may be a peptide sequence or a naturally occurring sequence. The solubility enhancing sequence may be selected from for example maltose binding protein (MBP), Small Ubiquitin-like Modifier (SUMO), Glutathione S-transferase (GST) or thioredoxin (TRX). The tags may be attached to either the C or N terminus. Any example of a solubility enhancer may be used. A list of possible proteins is shown below. Any sequence selected from the list below may be chosen:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation factor IF2 | IF2-domain 1 |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension sequence | NEXT |

Any fluorescent protein may be used. The term GFP is used herein to describe a group of green fluorescent proteins from different organisms including the proteins sfGFP, GFP, eGFP, ccGFP, deGFP, frGFP. The fluorescent protein may be sfGFP, GFP, eGFP, ccGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀. The peptide tag may be CFAST₁₁ or CFAST₁₀ and the further polypeptide CFAST in the presence of a hydroxybenzylidene rhodanine analog. The peptide tag may be ccGFP₁₁ and the further polypeptide ccGFP₁₋₁₀.

Disclosed is a method for the improved soluble yield of an expressed protein of interest (POI) in a cell-free system by expressing a protein of interest (POI) in the presence of a solubility enhancement moiety where the POI has a binding sequence which binds to the solubility enhancement moiety, wherein the solubility enhancement moiety comprises a component of a fluorescent protein and a moiety selected those in the table above.

The improved solubility of the detector protein enhances the soluble expression yield of the POI.

The fluorescent protein may be GFP. The fluorescent protein may be sfGFP. The fluorescent protein may be ccGFP. The solubility enhancement moiety may comprise ccGFP₁₋₁₀ and MBP.

The protein may be assembled and thereby become fluorescent as a result of the expressed protein binding with the binding partner. The affinity interaction results in the two sub-components of the fluorescent protein being near enough to each other to bind and induce fluorescence.

The complementary GFP₁₁ peptide amino acid sequence tag could be the following:

1.
(SEQ ID NO: 2)
KRDHMVLLEFVTAAGITGT
2.
(SEQ ID NO: 3)
KRDHMVLHEFVTAAGITGT
3.
(SEQ ID NO: 4)
KRDHMVLHESVNAAGIT
4.
(SEQ ID NO: 5)
RDHMVLHEYVNAAGIT
5.
(SEQ ID NO: 6)
GDAVQIQEHAVAKYFTV
6.
(SEQ ID NO: 7)
GDTVQLQEHAVAKYFTV
7.
(SEQ ID NO: 8)
GETIQLQEHAVAKYFTE

Properties of the expressed protein may be characterised on the device. An initial screen may be based on the level of soluble expression by measuring fluorescence formed on complementation of a detector with the expressed sequence. The protein may remain fluorescent during immobilisation, at which point the level of affinity purification can be determined.

Disclosed is a method of taking multiple droplets having a protein with a GFP₁₁ tag and monitoring changes in the level of the GFP₁₁ tag in order to determine protein expression yield.

The methods can measure aggregation over time by measuring solubility over time. For example,
1. A mother droplet expresses the POI.
2. The mother droplet is split into multiple daughter droplets.
3. A droplet containing detector is added to daughter droplet 1 at time 1.
4. Another droplet containing detector is added to daughter droplet 2 at time-2, wherein time-2 is greater than time-1.
5. The complementations from (3) and (4) are run for the same period of time each.
6. The fluorescence is measured from both droplets and compared. Apparent protein soluble yield is measured from the difference in fluorescence signal.

Once the protein is expressed the expression can be stopped by addition of chemical reagents to prevent further expression. The assay for stability should be independent of further expression, as increases in the amount of protein by further protein may bias the measurements of stability. For example the detector solution can be mixed with an agent that arrests protein synthesis, such as a Mg++ chelator (EDTA) in order to arrest and detect all in one. Alternatively the chelator/EDTA can be introduced first to all daughter droplets and the detector introduced at different time points.

In addition to screening for the best conditions for expression, the method can also be used to screen the best conditions for stability of particular proteins. A protein of interest can be made with a variety of different amino acid appendages acting as stability agents. The expressed amino acids can be exposed to a variety of different times, temperatures or environments and the amount of remaining protein material determined. Thus as well as screening for efficient synthesis, efficient stability conditions can also be identified. Once identified, the optimal conditions can be used for scale-up and purification.

The screening and analysis can be performed in liquid reagent volumes, for example in microtitre plates or strip-tubes. Reagent volumes can be split such that portions are tested and portions retained for further use.

Such screening, characterising and purification can all be performed on a single device, which may be a digital microfluidic device. The term digital microfluidic device refers to a device having a two-dimensional array of planar microelectrodes. The term digital microfluidic device excludes any devices simply having droplets in a flow of oil in a channel. The droplets are moved over the surface by electrokinetic forces by activation of particular electrodes. Upon activation of the electrodes the dielectric layer becomes less hydrophobic, thus causing the droplets to spread onto the surface. A digital microfluidic (DMF) device set-up is known in the art, and depends on the substrates used, the electrodes, the configuration of those electrodes, the use of a dielectric material, the thickness of that dielectric material, the hydrophobic layers, and the applied voltage.

The droplets can be aqueous droplets. The droplets can contain an oil immiscible organic solvent such as for example DMSO. The droplets can be a mixture of water and solvent, providing the droplets do not dissolve into the bulk oil.

Digital microfluidics (DMF) refers to a two-dimensional planar surface platform for lab-on-a-chip systems that is based upon the manipulation of microdroplets. Droplets can be dispensed, moved, stored, mixed, reacted, or analyzed on a platform with a set of insulated electrodes. Digital microfluidics can be used together with analytical analysis procedures such as mass spectrometry, colorimetry, electrochemical, and electrochemiluminescence.

The droplet can be moved using any means of electrokinesis. The aqueous droplet can be moved using electrowetting-on-dielectric (EWoD). Electrowetting on a dielectric (EWoD) is a variant of the electrowetting phenomenon that is based on dielectric materials. During EWoD, a droplet of a conducting liquid is placed on a dielectric layer with insulating and hydrophobic properties. Upon activation of the electrodes the dielectric layer becomes less hydrophobic, thus causing the droplet to spread onto the surface.

The electrical signal on the EWoD or optically-activated amorphous silicon (a-Si) EWoD device can be delivered through segmented electrodes, active-matrix thin-film transistors or digital micromirrors. Optically-activated s-Si EWoD devices are well known in the art for actuating droplets (J. Adhes. Sci. Technol., 2012, 26, 1747-1771).

A source of supplemental oxygen can be supplied to the droplets. For example droplets or gas bubbles containing gaseous or dissolved oxygen can be merged with the aqueous droplets during the protein expression. Alternatively the source of oxygen can be a molecular source which releases oxygen. Alternatively the droplets can be moved to an air/liquid boundary to enable increased diffusion of oxygen from a gaseous environment. Alternatively the oil can be oxygenated.

The droplet can be formed before entering the microfluidic device and flowed into the device. Alternatively the droplets can be merged on the device. Included is a method comprising merging a first droplet containing a nucleic acid template such as a plasmid with a second droplet containing a cell-free system having the components for protein expression to form the droplet.

The droplets can be actuated on a hydrophobic surface on the digital microfluidic device (ACS Nano 2018, 12, 6, 6050-6058). The hydrophobic surface can be a hydrophobic surface such as polytetrafluoroethylene (PTFE), Teflon AF (DuPont Inc), CYTOP (AGC Chemicals Inc), or FluoroPel (Cytonix LLC). The hydrophobic surface may be modified in such a way to reduce biofouling, especially biofouling resulting from exposure to CFPS reagents or nucleic acid reagents. The hydrophobic surface may also be superhydrophobic, such as NeverWet (NeverWet LLC) or Ultra-Ever Dry (Flotech Performance Systems Ltd). Superhydrophobic surfaces prevent biofouling compared with typical fluorocarbon-based hydrophobic surfaces. Superhydrophobic surfaces thus prolong the capability of digital microfluidic devices to move CFPS droplets and general solutions containing biopolymers (RSC Adv., 2017, 7, 49633-49648). The hydrophobic surface can also be a slippery liquid infused porous surface (SLIPS), which can be formed by infusing Krtox-103 oil (DuPont) with porous PTFE film (Lab Chip, 2019, 19, 2275).

For electrowetting on dielectrics (EWoD), the change in contact angle of reagent upon the application of electric potential is an inverse function of surface tension. Thus, for low voltage EWoD operations, reduction in surface tension is achieved by addition of surfactants to reagents, which for CFPS reactions means to the lysate and to the DNA. This results in a dilution of the lysate, and it has been seen, in experiments, that diluting the lysate results in a decrease in expression level of the protein of interest. Thus performing CFPS on DMF where the surfactants are added to the solutions being moved will necessarily result in a dilution of the lysate and thus a decrease in the level of protein expression. In addition to being a problem in its own right, this further complicates extrapolation of on-DMF results to in-tube predictions of protein yield. An additional detriment of having to add surfactants to the samples is that this increases the time required for sample preparation, as well as increasing the potential for inconsistent results due to ‘user error,’ as there is more handling of reagents. An additional detriment of having to add surfactants to the samples is that certain downstream operations are hindered. For example, if a protein of interest is expressed in a cell-free system with a GFP₁₁ (or similar) peptide tag, it's downstream complementation with a GFP₁₋₁₀ detector polypeptide is hindered in the presence of surfactant.

Rather than adding surfactants to the aqueous sample, it is instead possible to add surfactant, such as Span85 (sorbitan trioleate), to the oil. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be nonionic, anionic, cationic, amphoteric. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution and without adding aqueous surfactant. It has been shown that surfactants reduce the efficiency of some detection systems, including but not limited to the split GFP system, so removing surfactants from the reagent mix and instead adding them to the oil can be beneficial.

Disclosed is a method of taking multiple droplets having a protein with a GFP₁₁ tag and monitoring changes in the level of the GFP₁₁ tag in order to determine protein stability. Disclosed is a method comprising expressing proteins in droplets on a digital microfluidic device having a two-dimensional array of planar microelectrodes wherein the proteins have a GFP₁₁ peptide amino sequence tag, holding the droplets at a fixed temperature and monitoring the droplets for changes in the level of the amount of the GFP 11 peptide amino sequence tag in the droplets.

### Devices

The manipulation of droplets by the application of electrical potential can be achieved on electrodes covered with an insulator or a dielectric or a series of insulators or dielectrics. Droplet manipulation as a result of an applied electrical potential is known as electrowetting. Electrowetting occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semi-conductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

$$cos θ - cos {θ}_{0} = ( 1 / 2 γ LG ) c . {V}^{2}$$

where θ₀ is the contact angle when the electric field across the interfacial layer is zero, γLG is the liquid-gas tension, c is the specific capacitance (given as εᵣ. ε₀/t, where εᵣ is dielectric constant of the insulator/dielectric, ε₀ is permittivity of vacuum, t is thickness) and V is the applied voltage or electrical potential. The change in contact angle (inducing droplet movement) is thus a function of surface tension, electrical potential, dielectric thickness, and dielectric constant.

When a droplet is actuated by EWoD, there are two opposing sets of forces that act upon it: an electrowetting force induced by electric field and resistant forces that include the drag forces resulting from the interaction of the droplet with filler medium and the contact line friction (ref). The minimum voltage applied to balance the electrowetting force with the sum of all drag forces (threshold voltage) is variably determined by the thickness-to-dielectric contact ratio of the insulator/dielectric, (t/εᵣ)^{1/2}. Thus, to reduce actuation voltage, it is required to reduce (t/εᵣ)^{1/2} (i.e., increase dielectric constant or decrease insulator/dielectric thickness). To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages >100V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion. The driving voltage for TFTs or optically-activated a-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWoD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP. The thickness of this material as a hydrophobic coating on the dielectric is typically <100 nm and can have defects in the form of pinholes or a porous structure; hence, it is particularly important that the insulator/dielectric is pinhole free to avoid electrical shorting. Teflon has also been used as an insulator/dielectric, but it has higher voltage requirements due to its low dielectric constant and the thickness required to make it pinhole free. Other hydrophobic insulator/dielectric materials can include polymer-based dielectrics such as those based on siloxane, epoxy (e.g. SU-8), or parylene (e.g., parylene N, parylene C, parylene D, or parylene HT). Due to minimal contact angle hysteresis and a higher contact angle with aqueous solutions, Teflon is still used as a hydrophobic topcoat on these insulator/dielectric polymers. However, there are difficulties in reliably producing <1 micron pinhole-free coatings of parylene or SU-8; thus, the thickness of these materials is typically kept at 2-5 microns at the cost of increased voltage requirements for electrowetting. It has also been reported that traditional EWoD devices with parylene C are easily broken and unstable for repeated droplet manipulation with cell culture medium. Multi-layer insulator devices deposited with metal-oxide and parylene C films have been used to produce a more robust insulator/dielectric and enable operations with lower applied voltages. Inorganic materials, such metal oxides and semiconductor oxides, commonly used in the CMOS industry as “gate dielectrics”, have been used as insulator/dielectric for EWoD devices. They offer the advantage of utilizing standard cleanroom processes for thin film depositions (<100 nm). These materials are inherently hydrophilic, requiring an additional hydrophobic coating, and can be prone to pinhole formation as a result of thin film layer deposition process. Together with the need for lower voltage operations of EWoD, recent developmental work has focused on (1) using materials with improved dielectric properties (e.g., using high-dielectric constant insulators/dielectrics), (2) optimizing the fabrication process to make the insulator/dielectric pinhole free to avoid dielectric breakdown.

Operation of EWoD devices suffers from contact angle saturation and hysteresis, which is believed to be brought about by either one or combination of these phenomena: (1) entrapment of charges in the hydrophobic film or insulator/dielectric interface, (2) adsorption of ions, (3) thermodynamic contact angle instabilities, (4) dielectric breakdown of dielectric layer, (5) the electrode-electrode-insulator interface capacitance (arising from the double layer effect), and (6) fouling of the surface (such as by biomacromolecules). One of the adverse effects of this hysteresis is reduced operational lifetime of the EWoD-based device.

Contact angle hysteresis is believed to be a result of charge accumulation at the interface or within the hydrophobic insulator after several operations. The required actuation voltage increases due to this charging phenomenon resulting in eventual catastrophic dielectric breakdown. The most probable explanation is that pinholes at the insulator/dielectric may allow the liquid to come into contact with the electrode causing electrolysis. Electrolysis is further facilitated by pinhole-prone or porous hydrophobic insulators.

Most of the studies to understand contact angle hysteresis on EWoD have been conducted on short time scales and with low conductivity solutions. Long duration actuations (e.g., >1 hour) and high conductivity solutions (e.g., 1 M NaCl) could produce several effects other than electrolysis. The ions in solution can permeate through the hydrophobic coat (under the applied electric field) and interact with the underlying insulator/dielectric. Ion permeation can result in (1) change in dielectric constant due to charge entrapment (which is different from interfacial charging) and (2) change in surface potential of a pH sensitive metal oxide. Both can result in reduction of electrowetting forces to manipulate aqueous droplets, leading to contact angle hysteresis. The inventors have previously found that the damage from high conductivity solutions reduces or disables electrowetting on electrodes by inhibiting the modulation of contact angle when an electric field is applied.

An electrokinetic device includes a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes.

The dielectric layer may comprise silicon dioxide, silicon oxynitride, silicon nitride, hafnium oxide, yttrium oxide, lanthanum oxide, titanium dioxide, aluminum oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick. Combinations of more than one material may be used, and the dielectric layer may comprise more than one sublayer that may be of different materials.

The conformal layer may comprise a parylene, a siloxane, or an epoxy. It may be a thin protective parylene coating in between the insulating dielectric and the hydrophobic coating. Typically, parylene is used as a dielectric layer on simple devices. In this invention, the rationale for deposition of parylene is not to improve insulation/dielectric properties such as reduction in pinholes, but rather to act as a conformal layer between the dielectric and hydrophobic layers. The inventors find that parylene, as opposed to other similar insulating coatings of the same thickness such as PDMS (polydimethylsiloxane), prevent contact angle hysteresis caused by high conductivity solutions or solutions deviating from neutral pH for extended hours. The conformal layer may be between 10 nm and 100 μm thick. The conformal layer may be between 100 nm and 300 nm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nanocoating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The functional coating may include a dielectric layer comprising silicon nitride, a conformal layer comprising parylene, and a hydrophobic layer comprising an amorphous fluoropolymer. This has been found to be a particularly advantageous combination.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

The second substrate may also comprise a second hydrophobic layer disposed on the second electrode. The first and second substrates may be disposed so that the hydrophobic layer and the second hydrophobic layer face each other, thereby defining the electrokinetic workspace between the hydrophobic layers.

The method is particularly suitable for aqueous droplets with a volume of 1 μL or smaller.

The EWoD-based devices shown and described below are active matrix thin film transistor devices containing a thin film dielectric coating with a Teflon hydrophobic top coat. These devices are based on devices described in the E Ink Corp patent filing on “Digital microfluidic devices including dual substrate with thin-film transistors and capacitive sensing”, US patent application no 2019/0111433, incorporated herein by reference.

Described herein are electrokinetic devices, including:
a first substrate having a matrix of electrodes, wherein each of the matrix electrodes is coupled to a thin film transistor, and wherein the matrix electrodes are overcoated with a functional coating comprising:
a dielectric layer in contact with the matrix electrodes,
a conformal layer in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;
Described herein is an electrokinetic device, including:
a first substrate having a matrix of electrodes, wherein each of the matrix electrodes is coupled to a thin film transistor, and wherein the matrix electrodes are overcoated with a functional coating comprising:
one or more dielectric layer(s) comprising silicon nitride, hafnium oxide or aluminum oxide in contact with the matrix electrodes,
a conformal layer comprising parylene in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;
The electrokinetic devices as described may be used with other elements, such as for example devices for heating and cooling the device or reagent cartridges for the introduction of reagents as needed.

“Droplet” refers to a volume of liquid that electrowets a hydrophobic surface and is at least partially bounded by carrier fluid and/or, in some instances, a gas or gaseous mixture such as ambient air. For example, a droplet may be completely surrounded by carrier fluid or may be bounded by carrier fluid and one or more surfaces of an EWoD device. Droplets may take a wide variety of shapes; non-limiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more working surface of an EWoD device. Droplets may include typical polar fluids such as water, as is the case for aqueous or non-aqueous compositions, or may be mixtures or emulsions including aqueous and non-aqueous components. Droplets may also include dispersions and suspensions, for example magnetic beads in an aqueous solvent. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes.

“Droplet operation” refers to any manipulation of one or more droplets on a microfluidic device. A droplet operation may, for example, include: loading a droplet into the DMF device; dispensing one or more droplets from a source reservoir; splitting, separating or dividing a droplet into two or more droplets; moving a droplet from one location to another in any direction; merging or combining two or more droplets into a single droplet; diluting a droplet; mixing a droplet; agitating a droplet; deforming a droplet; holding a droplet in position; incubating a droplet; heating a droplet; vaporizing a droplet; cooling a droplet; disposing of a droplet; transporting a droplet out of a microfluidic device; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations includes but is not limited to microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles.

### Experimental & Results

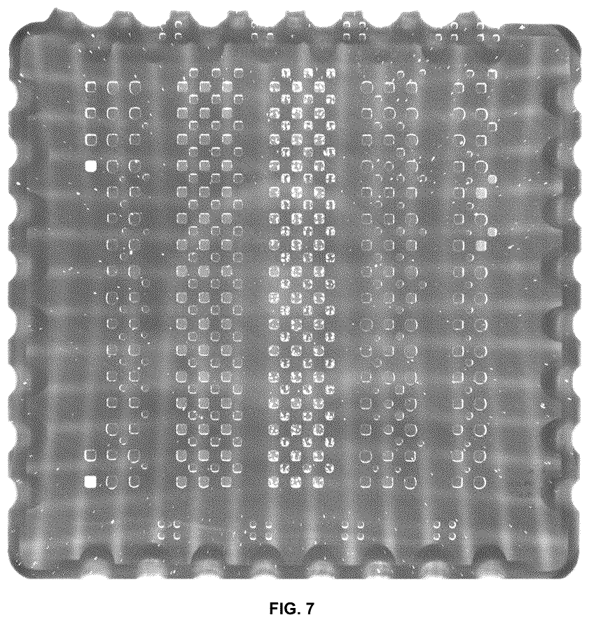

FIG. 5 shows the experimental result from 24 different proteins expressed in a reconstituted cell-free protein synthesis system in droplets on an electrowetting on dielectric (EWoD) device. Each construct contains a GFP₁₁ tag. In the rows marked Screen, the GFP₁₋₁₀ detector species is present from the start of expression. The rows marked Endpoint shows the fluorescence signal from 10 hours expression in the absence of GFP₁₋₁₀ detector species followed by 5 hours complementation with the GFP₁₋₁₀ detector species. This experiment showed significant differences between expression/complementation in Screen BioInk compared to Endpoint detection. The detected protein clusters formed after expression only with endpoint detection mean that the protein aggregated after expression, thereby lowering the soluble yield. It is evident from the image the presence of aggregates for several constructs, indicating the likely presence of protein aggregates. The level of aggregation enables identification of conditions which are worthy of further testing, and identification of conditions having high level of aggregated protein from which further purification is unlikely to give material.

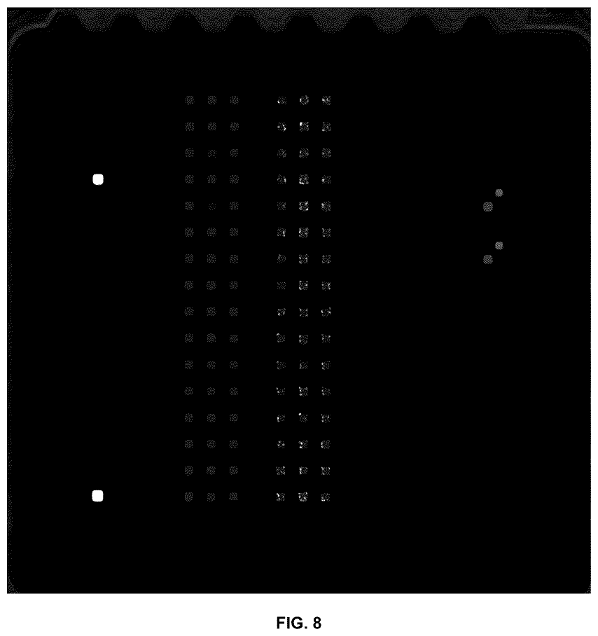

FIGS. 6-8 show expression results from two proteins expressed in a reconstituted cell-free protein synthesis system in droplets on an electrowetting on dielectric (EWoD) device. 8 different cell-free expression blends are tested for each of the proteins. FIG. 6 shows a white light image after 10 h expression. FIG. 7 shows a white light image after addition of detector protein (further 5 h). FIG. 8 shows a fluorescent (blue light) image after addition of detector protein. For a soluble protein (DHFR) (left side of array) the droplets appear uniform in the three images as the expressed protein does not appear as clumps, no clumping or aggregation can be seen in any of the 3 images. In the central 3 columns of the image after the expression (FIG. 6) clusters of protein (P17_IFN) can be detected in the optical image. The clusters remains detectable in the white light images after the detector protein has been added (FIG. 7). The fluorescence images (FIG. 8) show clusters of fluorescence within the droplets, indicating the presence of protein aggregates. Droplets having a high level of turbidity in the white light images also have a high level of signal from bond detector protein, indicating that protein can be detected either using white light or fluorescence measurements.

We claim:

1. A method for assessing the aggregation propensity of expressed proteins comprising:
a. expressing a protein of interest having a binding tag that upon binding to a detector moiety generates a fluorescent signal;
b. contacting with a detector moiety to generate a fluorescent signal upon binding to the tag; and
c. measuring the homogeneity of the fluorescent signal generated upon binding of the detector moiety to the binding tag to determine the level of aggregation of the protein of interest.

2. The method according to claim 1, wherein the fluorescent signal is measured using a thin film.

3. The method according to claim 1, wherein the fluorescent signal is measured in a capillary.

4. The method according to claim 1, wherein the fluorescent signal is measured using flow cytometry.

5. The method according to claim 1, wherein the fluorescent signal is measured using a low path length cuvette.

6. The method according to claim 1, wherein the protein is expressed in droplets.

7. The method according to claim 6, wherein the droplets are on a microfluidic device.

8. The method according to claim 7, wherein the microfluidic device contains channels for flowing droplets.

9. The method according to claim 7, wherein the device is a digital microfluidic device.

10. The method according to claim 7, wherein the droplets are spread on a surface by increasing the surface energy of the surface.

11. The method according to claim 1, wherein the protein is expressed in a cell-free protein synthesis system.

12. The method according to claim 1, wherein the ratio of soluble and insoluble POI is determined.

13. The method according to claim 1, wherein the degree of aggregation is measured by counting the number of aggregates, the area of the aggregates, the intensity of the aggregates or by using a measurement of dispersion.

14. The method according to claim 1, wherein the binding tag contains four or more amino acids.

15. The method according to claim 1, wherein the detector moiety is comprises a component of a fluorescent protein.

16. The method according to claim 15, wherein the detector moiety comprises a component of a fluorescent protein and a further solubility enhancer selected from:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation factor IF2 | IF2-domain 1 |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension sequence | NEXT |

17. The method according to claim 15, wherein the protein of interest has a ccGFP₁₁ tag and the detector moiety comprises ccGFP₁₋₁₀ and MBP.

18. The method according to claim 1, wherein the expression is performed for at least 3 hours before the detector moiety which binds to the POI is added.

19. The method according to claim 1, wherein the expression is performed using cell-free lysates or using assembled components for transcription and translation in a system of purified recombinant elements (PURE).

20. The method according to claim 9, wherein the digital microfluidic device comprises an oil-filled or humidified gaseous environment, wherein the humidified gaseous environment is achieved by enclosing or sealing the digital microfluidic device and providing on-board reagent reservoirs.
